(19) United States
(12) Patent Application Publication
(10) Pub. No.: US 2017/0188899 A1
(43) Pub. Date: Jul. 6, 2017

(54) Device to Collect Raman Photons for Non-Invasive Glucose Detector

(71) Applicant: xuan zhong ni, santa rosa, CA (US)

(72) Inventor: Xuan Zhong Ni, Santa Rosa, CA (US)

(21) Appl. No.: 14/987,427

(22) Filed: Jan. 4, 2016

(51) Int. Cl. A61B 5/1455; A61B 5/145; A61B 5/00; G01N 21/65

(52) U.S. Cl. CPC A61B 5/1455; G01N 21/65; A61B 5/14532; A61B 5/6826

(57) ABSTRACT

The present invention relates to a device which uses optical fibers to form a tube hat for a finger tip to collect all Raman photons.

### BACKGROUND OF THE INVENTION

In Raman scattering the photons were scattered inelastically, resulting the light of the scatted photons in all direction. This is one reason to cause the weak Raman signals. Trying to collect all scattered Raman photons will increase the strength of the Raman signals.

U.S. Pat. No. 8,355,767 B2 (Hunter et al.) filed on Apr. 27, 2006, disclosed a Raman spectroscopy for non-invasive glucose measurements which use a side-looking parabolic mirror to collect Raman light.

None of the prior art methods used multiple optical fibers surround a finger tip.

### SUMMARY OF THE INVENTION

The present invention relates to a device which uses optical fibers to form a tube hat for a finger tip to collect all Raman photons.

To perform accurate measurements of glucose requires improvements in the signal to noise ratio due to the Weak Raman signals. By using optical fibers forming a tube hat for the finger tip under detecting it will increase the number of optical fibers used for collecting Raman photons and will substantially improve the signal to noise ratio.

### BRIEF DESCRIPTION OF THE DRAWINGS

FIG. 1 is a schematic illustration of a light delivery and collection device in accordance with a preferred embodiment of the invention.

### DETAILED DESCRIPTION OF THE INVENTION

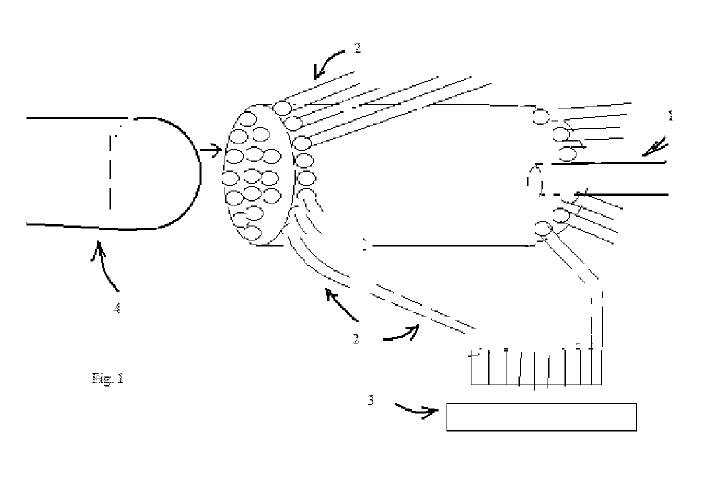

Raman photons were collected by a device using optical fibers to form a tube hat of a finger tip. The device shown in FIG. 1 used a light source 1 as the Raman excitation source. The scattered Raman photons from the finger tip 4 were collected by the optical fiber bundle 2 leading to the spectrometer entrance slit 3.

I claim:

1. A Raman photon collecting device using optical fibers with ends of fibers forming a tube hat for a finger tip.
